(19) United States
(12) Patent Application Publication
(10) Pub. No.: US 2021/0397989 A1
(43) Pub. Date: Dec. 23, 2021

(54) ARTIFICIAL INTELLIGENCE BASED SYSTEM FOR PATTERN RECOGNITION IN A SEMI-SECURE ENVIRONMENT

(71) Applicant: BANK OF AMERICA CORPORATION, Charlotte, NC (US)

(72) Inventors: Kyle Mayers, Charlotte, NC (US); George Albero, Charlotte, NC (US); Jinna Zevulun Kim, Charlotte, NC (US); Philip Lone Mintac, Charlotte, NC (US); Dustin Paul Stocks, Stallings, NC (US)

(73) Assignee: BANK OF AMERICA CORPORATION, Charlotte, NC (US)

(21) Appl. No.: 16/908,061

(22) Filed: Jun. 22, 2020

(51) Int. Cl. G06N 5/04; G06N 20/00; G06Q 40/02; G06Q 50/26; H04L 29/08; G08B 25/00

(52) U.S. Cl. CPC G06N 5/04; G06N 20/00; G06Q 40/02; G06Q 50/265; G16H 40/67; H04L 67/18; H04L 67/12; G08B 25/006; H04L 67/22

(57) ABSTRACT

Embodiments of the present invention provide a system monitoring and identifying real-time indicators and patterns to predict occurrence of real-time events. The system is configured for continuously gathering and monitoring real-time data from one or more monitoring devices associated with the entity, identifying that at least one user device is within a predetermined distance and automatically transmit a prompt to the user device to connect to a beacon, identifying that at least one user associated with the at least one user device has connected to the beacon, continuously monitoring the at least one user device in real-time, identifying occurrence of a potential real-time event based on continuously monitoring the at least one user device and the real-time data from the one or more monitoring devices, via an artificial intelligence engine.

### BACKGROUND

Conventional systems do not have the capability to monitor and identify real-time indicators and patterns to predict occurrence of real-time events. As such, there exists a need for a system that monitors and identifies real-time indicators and patterns to predict occurrence of real-time events.

### BRIEF SUMMARY

The following presents a summary of certain embodiments of the invention. This summary is not intended to identify key or critical elements of all embodiments nor delineate the scope of any or all embodiments. Its sole purpose is to present certain concepts and elements of one or more embodiments in a summary form as a prelude to the more detailed description that follows.

Embodiments of the present invention address the above needs and/or achieve other advantages by providing apparatuses (e.g., a system, computer program product and/or other devices) and methods for monitoring and identifying real-time indicators and patterns to predict occurrence of real-time events. The system embodiments may comprise one or more memory devices having computer readable program code stored thereon, a communication device, and one or more processing devices operatively coupled to the one or more memory devices, wherein the one or more processing devices are configured to execute the computer readable program code to carry out the invention. In computer program product embodiments of the invention, the computer program product comprises at least one non-transitory computer readable medium comprising computer readable instructions for carrying out the invention. Computer implemented method embodiments of the invention may comprise providing a computing system comprising a computer processing device and a non-transitory computer readable medium, where the computer readable medium comprises configured computer program instruction code, such that when said instruction code is operated by said computer processing device, said computer processing device performs certain operations to carry out the invention.

In some embodiments, the present invention continuously gathers and monitors real-time data from one or more monitoring devices associated with the entity, identifies that at least one user device is within a predetermined distance and automatically transmits a prompt to the user device to connect to a beacon, identifies that at least one user associated with the at least one user device has connected to the beacon, continuously monitors the at least one user device in real-time, identifies occurrence of a potential event based on continuously monitoring the at least one user device and the real-time data from the one or more monitoring devices, via an artificial intelligence engine.

In some embodiments, the present invention in response to identifying occurrence of a potential event, trigger one or more actions.

In some embodiments, the one or more actions comprises at least one of automatically controlling one or more entity systems within a facility associated with the entity, automatically generating and transmitting one or more alerts to the one or more third party entities, automatically transmitting the real-time data from the one or more monitoring device to the one or more third party entities, and controlling network associated with network communications of the at least one user device and the one or more entity systems located within the facility.

In some embodiments, identifying the occurrence of the potential event comprises identifying one or more patterns that are similar to historical patterns from at least one of (i) data from the at least one user device and (ii) the real-time data from the one or more monitoring devices, wherein the historical patterns are associated with historical events.

In some embodiments, the one or more patterns are associated with type of movements of entity users and the at least one user, type of clothing, type of activity, and type of biosensing signals from the entity users and the at least one user.

In some embodiments, the present invention identifies that the at least one user device is not within the predetermine distance and automatically disconnects the at least one user device from the beacon.

In some embodiments, the present invention the one or more monitoring devices comprises at least one of one or more sensors, biosensors, and activity trackers.

The features, functions, and advantages that have been discussed may be achieved independently in various embodiments of the present invention or may be combined with yet other embodiments, further details of which can be seen with reference to the following description and drawings.

### BRIEF DESCRIPTION OF THE DRAWINGS

Having thus described embodiments of the invention in general terms, reference will now be made the accompanying drawings, wherein:

FIG. 1 provides a block diagram illustrating a system environment monitoring and identifying real-time indicators and patterns to predict occurrence of real-time events, in accordance with an embodiment of the invention;

FIG. 2 provides a block diagram illustrating the entity system 200 of FIG. 1, in accordance with an embodiment of the invention;

FIG. 3 provides a block diagram illustrating a real-time pattern recognition system 300 of FIG. 1, in accordance with an embodiment of the invention;

FIG. 4 provides a block diagram illustrating the computing device system 400 of FIG. 1, in accordance with an embodiment of the invention; and

FIG. 5 provides a flowchart illustrating a process flow monitoring and identifying real-time indicators and patterns to predict occurrence of real-time events, in accordance with an embodiment of the invention.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

In some embodiments, an “entity” may be any organization that comprises one or more facilities that serves one or more users. In some specific embodiments, an “entity” as used herein may include any financial institutions such as commercial banks, thrifts, federal and state savings banks, savings and loan associations, credit unions, investment companies, insurance companies and the like.

As described herein, a “user” may be a customer of the entity. In some embodiments, where the entity is a financial institution, the user may be a customer that holds resource accounts with the financial institution.

As described herein, a “entity user” may be an employee of the entity, where the employee may be a full-time employee, contractor, sub-contractor, or the like. In some embodiments, where the entity is a financial institution, the entity user may be a teller. In some embodiments, the entity user may be any user that works within a facility (e.g., financial center) of the entity.

Many of the example embodiments and implementations described herein contemplate interactions engaged in by the user with a computing device and/or one or more communication devices and/or secondary communication devices. Furthermore, as used herein, the term “user computing device” or “mobile device” may refer to mobile phones, computing devices, tablet computers, wearable devices, smart devices and/or any portable electronic device capable of receiving and/or storing data therein.

A “user interface” is any device or software that allows a user to input information, such as commands or data, into a device, or that allows the device to output information to the user. For example, the user interface includes a graphical user interface (GUI) or an interface to input computer-executable instructions that direct a processing device to carry out specific functions. The user interface typically employs certain input and output devices to input data received from a user or to output data to a user. These input and output devices may include a display, mouse, keyboard, button, touchpad, touch screen, microphone, speaker, LED, light, joystick, switch, buzzer, bell, and/or other user input/output device for communicating with one or more users.

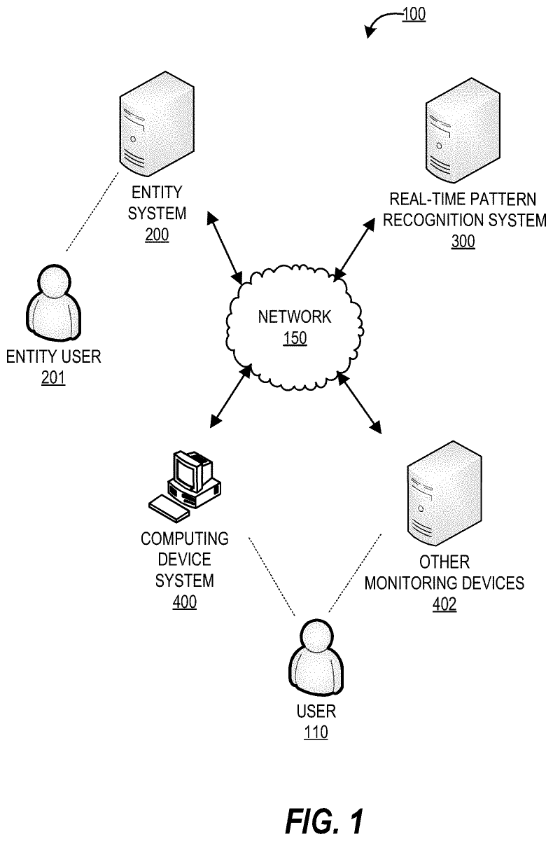

FIG. 1 provides a block diagram illustrating a system environment 100 monitoring and identifying real-time indicators and patterns to predict occurrence of real-time events, in accordance with an embodiment of the invention. As illustrated in FIG. 1, the environment 100 includes a real-time pattern recognition system 300, an entity system 200, a computing device system 400, and other monitoring devices 402. One or more users 110 may be included in the system environment 100, where the users 110 interact with the other entities of the system environment 100 via a user interface of the computing device system 400. In some embodiments, the one or more user(s) 110 of the system environment 100 may be customers of the entity associated with the entity system 200. One or more entity user(s) 201 may be employees of the entity that are allowed to use, control, and/or manage the entity system(s) 200. In some embodiments, the entity system(s) 200 may be any computing systems located within a facility of the entity. In some embodiments, the entity system(s) 200 may be any remote computing systems that are associated with the entity, but are not located within the facility.

The entity system(s) 200 may be any system owned by an entity and/or or otherwise controlled by the one or more entity users 201 to support or perform one or more operations associated with the entity. In some embodiments, the entity is a financial institution. In some embodiments, the entity is a non-financial institution.

The real-time pattern recognition system 300 is a system of the present invention for performing one or more process steps described herein. In some embodiments, the real-time pattern recognition system 300 may be an independent system. In some embodiments, the real-time pattern recognition system 300 may be a part of at least one of the entity system(s) 200.

The real-time pattern recognition system 300, the entity system 200, the computing device system 400, and the other monitoring devices 402 may be in network communication across the system environment 100 through the network 150. The network 150 may include a local area network (LAN), a wide area network (WAN), and/or a global area network (GAN). The network 150 may provide for wireline, wireless, or a combination of wireline and wireless communication between devices in the network. In one embodiment, the network 150 includes the Internet. In general, the real-time pattern recognition system 300 is configured to communicate information or instructions with the entity system 200, and/or the computing device system 400 across the network 150.

The computing device system 400 may be a system owned or controlled by the entity of the entity system 200 and/or the one or more users 110. As such, the computing device system 400 may be a computing device of the user 110. In general, the computing device system 400 communicates with the user 110 via a user interface of the computing device system 400, and in turn is configured to communicate information or instructions with the real-time pattern recognition system 300, and/or entity system 200 across the network 150. In some embodiments, the computing device system may be at least one of a mobile device, smart device, wearable device, or the like.

In some embodiments, the other monitoring devices 402 may include, but are not limited to visual devices (e.g., camera), IoT trackers, auditory devices (e.g., telephone), motion sensors, biosensors, activity trackers, or the like in a non-intrusive manner. The other monitoring devices may be owned and/or controlled by the entity of the entity system 200. In some embodiments, each of the other monitoring devices 402 may include a real-time pattern recognition application provided by the real-time pattern recognition system 300 to facilitate the capture of information associated with a facility of the entity.

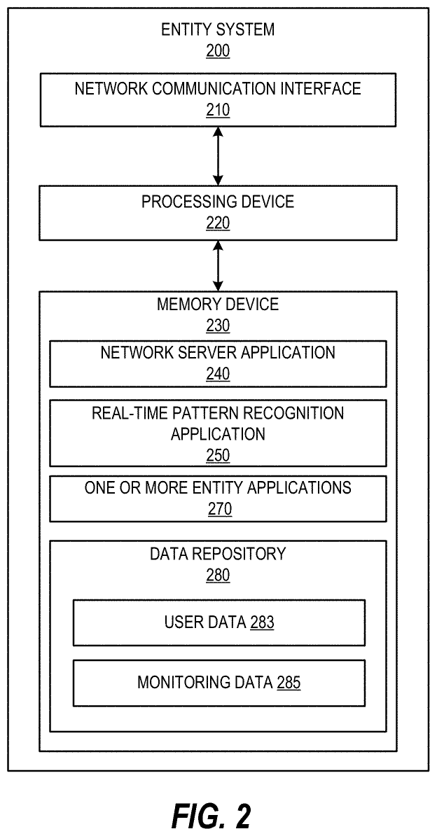

FIG. 2 provides a block diagram illustrating the entity system 200, in greater detail, in accordance with embodiments of the invention. As illustrated in FIG. 2, in one embodiment of the invention, the entity system 200 includes one or more processing devices 220 operatively coupled to a network communication interface 210 and a memory device 230.

It should be understood that the memory device 230 may include one or more databases or other data structures/repositories. The memory device 230 also includes computer-executable program code that instructs the processing device 220 to operate the network communication interface 210 to perform certain communication functions of the entity system 200 described herein. For example, in one embodiment of the entity system 200, the memory device 230 includes, but is not limited to, a real-time pattern recognition application 250, one or more entity applications 270, and a data repository 280 comprising information associated with user data 283 (e.g., customer data) and monitoring data 285. The computer-executable program code of the network server application 240, the real-time pattern recognition application 250, the one or more entity applications 270 to perform certain logic, data-extraction, and data-storing functions of the entity system 200 described herein, as well as communication functions of the entity system 200.

The network server application 240, the real-time pattern recognition application 250, and the one or more entity applications 270 are configured to store data in the data repository 280 or to use the data stored in the data repository 280 when communicating through the network communication interface 210 with the real-time pattern recognition system 300, the computing device system 400, and/or other monitoring devices 402 to perform one or more process steps described herein. In some embodiments, the entity system 200 may receive instructions from the real-time pattern recognition system 300 via the real-time pattern recognition application 250 to perform certain operations. The real-time pattern recognition application 250 may be provided by the real-time pattern recognition system 300. The one or more entity applications 270 may be any of the applications used, created, modified, facilitated, and/or managed by the entity system 200.

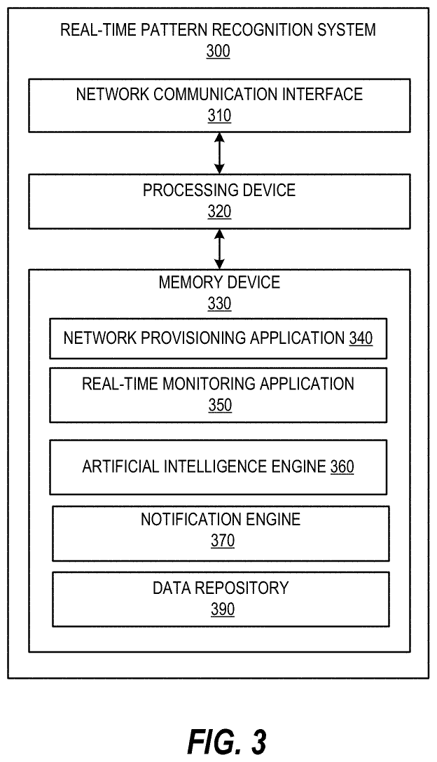

FIG. 3 provides a block diagram illustrating the real-time pattern recognition system 300 in greater detail, in accordance with embodiments of the invention. As illustrated in FIG. 3, in one embodiment of the invention, the real-time pattern recognition system 300 includes one or more processing devices 320 operatively coupled to a network communication interface 310 and a memory device 330. In certain embodiments, the real-time pattern recognition system 300 is operated by an entity, such as a financial institution. In some embodiments, the real-time pattern recognition system 300 may be an independent system. In alternate embodiments, the real-time pattern recognition system 300 may be a part of the entity system 200.

It should be understood that the memory device 330 may include one or more databases or other data structures/repositories. The memory device 330 also includes computer-executable program code that instructs the processing device 320 to operate the network communication interface 310 to perform certain communication functions of the real-time pattern recognition system 300 described herein. For example, in one embodiment of the real-time pattern recognition system 300, the memory device 330 includes, but is not limited to, a network provisioning application 340, a real-time monitoring application 350, an artificial intelligence engine 360, a notification engine 370, and a data repository 390 comprising data processed or accessed by one or more applications in the memory device 330. The computer-executable program code of the network provisioning application 340, the real-time monitoring application 350, the artificial intelligence engine 360, and the notification engine 370 may instruct the processing device 320 to perform certain logic, data-processing, and data-storing functions of the real-time pattern recognition system 300 described herein, as well as communication functions of the real-time pattern recognition system 300.

The network provisioning application 340, network provisioning application 340, the real-time monitoring application 350, the artificial intelligence engine 360, and the notification engine 370 are configured to invoke or use the data in the data repository 390 when communicating through the network communication interface 310 with the entity system 200, and/or the computing device system 400. In some embodiments, network provisioning application 340, the real-time monitoring application 350, the artificial intelligence engine 360, and the notification engine 370 may store the data extracted or received from the entity system 200, and the computing device system 400 in the data repository 390. In some embodiments, network provisioning application 340, the real-time monitoring application 350, the artificial intelligence engine 360, and the notification engine 370 may be a part of a single application stored in the memory device 330. The functions of the network provisioning application 340, the real-time monitoring application 350, the artificial intelligence engine 360, and the notification engine 370 are described in more detail in FIG. 6.

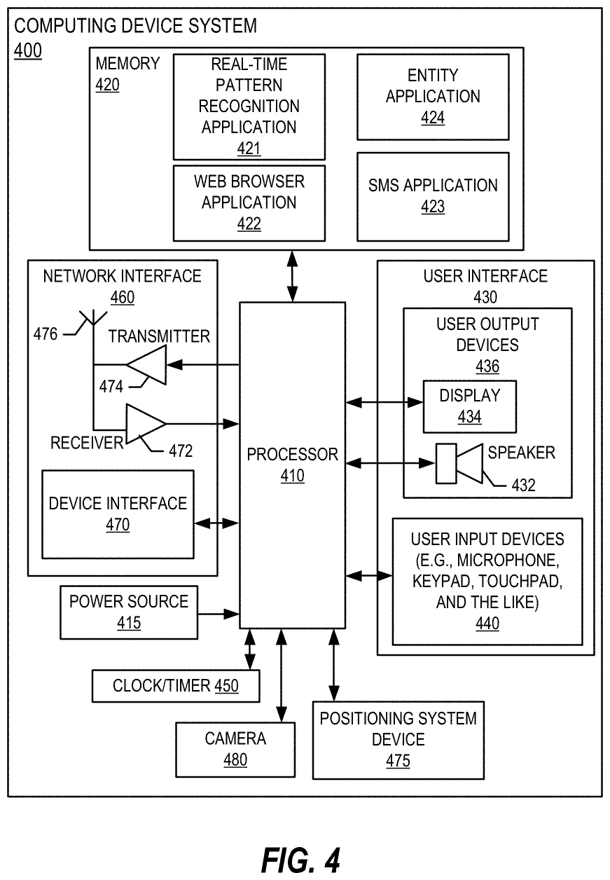

FIG. 4 provides a block diagram illustrating a computing device system 400 of FIG. 1 in more detail, in accordance with embodiments of the invention. However, it should be understood that a mobile telephone is merely illustrative of one type of computing device system 400 that may benefit from, employ, or otherwise be involved with embodiments of the present invention and, therefore, should not be taken to limit the scope of embodiments of the present invention. Other types of computing devices may include portable digital assistants (PDAs), pagers, mobile televisions, gaming devices, desktop computers, workstations, laptop computers, cameras, video recorders, audio/video player, radio, GPS devices, wearable devices, Internet-of-things devices, augmented reality devices, virtual reality devices, automated teller machine devices, electronic kiosk devices, or any combination of the aforementioned.

Some embodiments of the computing device system 400 include a processor 410 communicably coupled to such devices as a memory 420, user output devices 436, user input devices 440, a network interface 460, a power source 415, a clock or other timer 450, a camera 480, and a positioning system device 475. The processor 410, and other processors described herein, generally include circuitry for implementing communication and/or logic functions of the computing device system 400. For example, the processor 410 may include a digital signal processor device, a microprocessor device, and various analog to digital converters, digital to analog converters, and/or other support circuits. Control and signal processing functions of the computing device system 400 are allocated between these devices according to their respective capabilities. The processor 410 thus may also include the functionality to encode and interleave messages and data prior to modulation and transmission. The processor 410 can additionally include an internal data modem. Further, the processor 410 may include functionality to operate one or more software programs, which may be stored in the memory 420. For example, the processor 410 may be capable of operating a connectivity program, such as a web browser application 422. The web browser application 422 may then allow the computing device system 400 to transmit and receive web content, such as, for example, location-based content and/or other web page content, according to a Wireless Application Protocol (WAP), Hypertext Transfer Protocol (HTTP), and/or the like.

The processor 410 is configured to use the network interface 460 to communicate with one or more other devices on the network 150. In this regard, the network interface 460 includes an antenna 476 operatively coupled to a transmitter 474 and a receiver 472 (together a “transceiver”). The processor 410 is configured to provide signals to and receive signals from the transmitter 474 and receiver 472, respectively. The signals may include signaling information in accordance with the air interface standard of the applicable cellular system of the wireless network 152. In this regard, the computing device system 400 may be configured to operate with one or more air interface standards, communication protocols, modulation types, and access types. By way of illustration, the computing device system 400 may be configured to operate in accordance with any of a number of first, second, third, and/or fourth-generation communication protocols and/or the like.

As described above, the computing device system 400 has a user interface that is, like other user interfaces described herein, made up of user output devices 436 and/or user input devices 440. The user output devices 436 include a display 430 (e.g., a liquid crystal display or the like) and a speaker 432 or other audio device, which are operatively coupled to the processor 410. In some embodiments, the display 430 may a touch-screen display, where the user is capable of acting as an input device to the computing device system 400.

The user input devices 440, which allow the computing device system 400 to receive data from a user such as the user 110, may include any of a number of devices allowing the computing device system 400 to receive data from the user 110, such as a keypad, keyboard, touch-screen, touchpad, microphone, mouse, joystick, other pointer device, button, soft key, and/or other input device(s). The user interface may also include a camera 480, such as a digital camera.

The computing device system 400 may also include a positioning system device 475 that is configured to be used by a positioning system to determine a location of the computing device system 400. For example, the positioning system device 475 may include a GPS transceiver. In some embodiments, the positioning system device 475 is at least partially made up of the antenna 476, transmitter 474, and receiver 472 described above. For example, in one embodiment, triangulation of cellular signals may be used to identify the approximate or exact geographical location of the computing device system 400. In other embodiments, the positioning system device 475 includes a proximity sensor or transmitter, such as an RFID tag, that can sense or be sensed by devices known to be located proximate a merchant or other location to determine that the computing device system 400 is located proximate these known devices.

The computing device system 400 further includes a power source 415, such as a battery, for powering various circuits and other devices that are used to operate the computing device system 400. Embodiments of the computing device system 400 may also include a clock or other timer 450 configured to determine and, in some cases, communicate actual or relative time to the processor 410 or one or more other devices.

The computing device system 400 also includes a memory 420 operatively coupled to the processor 410. As used herein, memory includes any computer readable medium (as defined herein below) configured to store data, code, or other information. The memory 420 may include volatile memory, such as volatile Random Access Memory (RAM) including a cache area for the temporary storage of data. The memory 420 may also include non-volatile memory, which can be embedded and/or may be removable. The non-volatile memory can additionally or alternatively include an electrically erasable programmable read-only memory (EEPROM), flash memory or the like.

The memory 420 can store any of a number of applications which comprise computer-executable instructions/code executed by the processor 410 to implement the functions of the computing device system 400 and/or one or more of the process/method steps described herein. For example, the memory 420 may include such applications as a conventional web browser application 422, a real-time pattern recognition application 421, an SMS application 423, and an entity application 424. These applications also typically instructions to a graphical user interface (GUI) on the display 430 that allows the user 110 to interact with the entity system 200, the real-time pattern recognition system 300, and/or other devices or systems. The memory 420 of the computing device system 400 may comprise a Short Message Service (SMS) application 423 configured to send, receive, and store data, information, communications, alerts, and the like via the wireless telephone network 152.

The memory 420 can also store any of a number of pieces of information, and data, used by the computing device system 400 and the applications and devices that make up the computing device system 400 or are in communication with the computing device system 400 to implement the functions of the computing device system 400 and/or the other systems described herein.

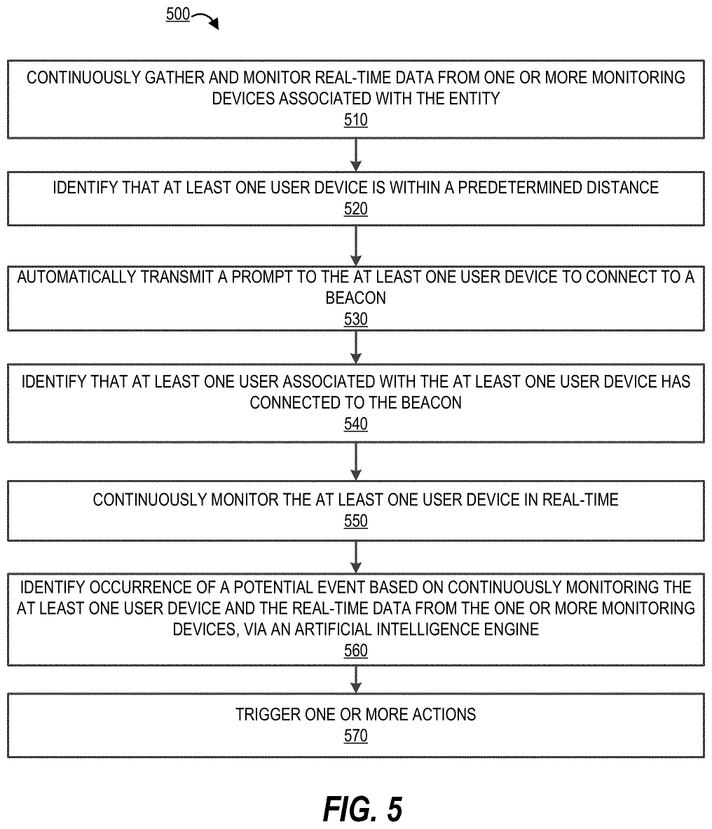

FIG. 5 provides a flowchart illustrating a process flow monitoring and identifying real-time indicators and patterns to predict occurrence of real-time events, in accordance with an embodiment of the invention.

As shown in block 510, the system continuously gathers and monitors real-time data from one or more monitoring devices associated with the entity. The one or more monitoring devices may comprise visual devices (e.g., video capturing device, camera, or the like), one or more sensors that may include, but are not limited to, motion sensors, biosensors, thermal sensors, or the like, auditory devices, wearable devices that are given to the entity users (e.g., tellers), and/or the like. In some embodiments, the one or more monitoring devices may comprise a wearable device that is provided to the entity user (e.g., employee) by the entity.

As shown in block 520, the system identifies that at least one user device is within a predetermined distance. The at least one user device may be a mobile phone, a smart watch, or any other wearable or portable device carried by the user. As shown in block 530, the system automatically transmits a prompt to the user device to connect to a beacon. The beacon may be a wireless transmitter that is owned, managed, operated, and/or controlled by the entity. In some embodiments, the beacon may be a wireless transmitter that transmits signals to smart devices that are in the vicinity. In an exemplary embodiment, the system may identify that at least one user is entering a facility of the entity and may prompt the at least one user device of the at least one user to connect to the beacon. In some embodiments, instead of a beacon, any other device that is capable of transmitting signals to user devices within the predetermined distance may be used.

As shown in block 540, the system identifies that at least one user associated with the at least one user device has connected to the beacon. As shown in block 550, the system continuously monitors the at least one user device in real-time. The system may monitor the user movements, user activity, or the like after identifying that the at least one user device is connected to the beacon. In some embodiments, the system may monitor the user activity only after receiving permission from the at least one user. In some embodiments, the system may identify that at least one user device is connected to the beacon and may compare at least one device identifier of the at least one user device with historical event data. As described herein, an “event” may be a misappropriation event (e.g., robbery), health related event, or the like. For example, the system may compare a device identifier or signature associated with the user device with stored signatures and/or identifiers that were involved in historical misappropriation events.

As shown in block 560, the system identifies occurrence of a potential event based on continuously monitoring the at least one user device and the real-time data from the one or more monitoring devices, via an artificial intelligence engine. In some embodiments, the system may identify one or more patterns from the real-time data captured from the one or more monitoring devices and data from the at least one user device to identify the occurrence of the potential event. The one or more patterns may be associated with type of movements, type of clothing, type of activity, type of biosensing signals, or the like. In one example, the system may identify a face mask that convers the entire face of a user entering the facility and identify that the user may be a potential bad actor. In another example, the system may identify a user entering the facility is wearing a set of clothing that is similar to the clothing used by a bad actor in a historical event and may identify that the user may be a potential bad actor. In another example, the system may identify that the health data from a biosensing signal associated with an entity user matches historical event data and may identify that an event is about to occur. In yet another example, the system may track and compare heartbeat or temperature of a user and/or an entity user with historical event data to predict occurrence of a health related event. The system may utilize any combination of the one or more patterns to identify occurrence of the potential event. In some embodiments, the system may identify the occurrence of a potential event based on predetermined indictors (e.g., threshold blood pressure, threshold heartbeat, threshold temperature, or the like).

In some embodiments, the system identifies or predicts the occurrence of the potential event based only on the real-time data captured from the one or more monitoring devices. In some embodiments, the system identifies or predicts the occurrence of the potential event based only on the data received from the at least one user device that is connected to the beacon. In some embodiments, the system identifies or predicts the occurrence of the potential event based on data received from third party systems (e.g., law enforcement agency), or system of the present invention used at a different facility. The system may receive data from a third party system about an event that had just occurred and the system monitors the real-time data from the one or more monitoring devices and/or the data associated with the at least one user device to look for similarities with the data received from the third party system. For example, the system may receive data associated with a misappropriation event that occurred within the last week and analyzes the received data to identify set of patterns and/or device identifiers associated with user devices that were present at a facility where the misappropriation event occurred. The system may then monitor the real-time data from the monitoring devices to look for the set of patterns and/or compare device identifiers that are connecting to the beacon at the current facility with the device identifiers received from the third party system or the system of the present invention used at a different facility.

As shown in block 570, the system triggers one or more actions. The one or more actions may include, but are not limited to, automatically controlling one or more entity systems within a facility associated with the entity, automatically generating and transmitting one or more alerts to the one or more third party entities (e.g., hospital, law enforcement agencies, or the like), automatically transmitting the real-time data from the one or more monitoring device (e.g., live feed) to the one or more third party entities, and controlling network associated with network communications of the at least one user device and the one or more entity systems located within the facility. The one or more actions implemented by the system may be based on identifying the type of the event (e.g., misappropriation event, health related event, or the like).

In one example, the system may identify occurrence of event and may alert authorities based on the type of the event (e.g., if the event is a health related event hospitals may be notified, if the event is a misappropriation event law enforcement agencies may be notified). In another example, the system may control (e.g., implement security measures, lock down systems, or the like) one or more entity systems (e.g., computers, ATM, kiosks, cash vaults, or the like) present within the facility after determining occurrence of a misappropriation event. In another example, the system may create a temporary cell tower to capture all outgoing and incoming network communications of user devices and entity systems after identifying occurrence of an event. In some cases, the system may transmit the network communications to one or more third party systems (e.g., law enforcement agencies). In some embodiments, this process flow may be applicable to a use case where a bad actor may be intimidating an elderly user to withdraw resources from an entity system (e.g., ATM).

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, and the like), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an object oriented, scripted or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational steps to be performed on the computer or other programmable apparatus to produce a computer-implemented process such that the code portions which execute on the computer or other programmable apparatus provide steps for implementing the functions/acts specified in the flowchart and/or block diagram block(s). Alternatively, computer program implemented steps or acts may be combined with operator or human implemented steps or acts in order to carry out an embodiment of the invention.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that steps of the processes described herein may be performed in orders different than those illustrated in the flowcharts. In other words, the processes represented by the blocks of a flowchart may, in some embodiments, be in performed in an order other that the order illustrated, may be combined or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1. A system monitoring and identifying real-time indicators and patterns to predict occurrence of real-time events, the system comprising:
at least one network communication interface;
at least one non-transitory storage device; and
at least one processing device coupled to the at least one non-transitory storage device and the at least one network communication interface, wherein the at least one processing device is configured to:
continuously gather and monitor real-time data from one or more monitoring devices associated with an entity;
identify that at least one user device is within a predetermined distance and automatically transmit a prompt to the at least one user device to connect to a beacon;
identify that at least one user associated with the at least one user device has connected to the beacon;
continuously monitor the at least one user device in real-time;
identify occurrence of a potential real-time event based on continuously monitoring the at least one user device and the real-time data from the one or more monitoring devices, via an artificial intelligence engine.

2. The system of claim 1, wherein the at least one processing device is configured to in response to identifying occurrence of the potential real-time event, trigger one or more actions.

3. The system of claim 2, wherein the one or more actions comprises at least one of:
automatically controlling one or more entity systems within a facility associated with the entity;
automatically generating and transmitting one or more alerts to the one or more third party entities;
automatically transmitting the real-time data from the one or more monitoring device to the one or more third party entities; and
controlling network associated with network communications of the at least one user device and the one or more entity systems located within the facility.

4. The system of claim 1, wherein identifying the occurrence of the potential real-time event comprises:
identifying one or more patterns that are similar to historical patterns from at least one of (i) data from the at least one user device and (ii) the real-time data from the one or more monitoring devices, wherein the historical patterns are associated with historical events.

5. The system of claim 4, wherein the one or more patterns are associated with type of movements of entity users and the at least one user, type of clothing, type of activity, and type of biosensing signals from the entity users and the at least one user.

6. The system of claim 1, wherein the at least one processing device is configured to:
identify that the at least one user device is not within the predetermine distance; and
automatically disconnect the at least one user device from the beacon.

7. The system of claim 1, wherein the one or more monitoring devices comprises at least one of one or more sensors, biosensors, and activity trackers.

8. A computer program product monitoring and identifying real-time indicators and patterns to predict occurrence of real-time events, the computer program product comprising a non-transitory computer-readable storage medium having computer executable instructions for causing a computer processor to perform the steps of:
continuously gathering and monitoring real-time data from one or more monitoring devices associated with an entity;
identifying that at least one user device is within a predetermined distance and automatically transmit a prompt to the at least one user device to connect to a beacon;
identifying that at least one user associated with the at least one user device has connected to the beacon;
continuously monitoring the at least one user device in real-time;
identifying occurrence of a potential real-time event based on continuously monitoring the at least one user device and the real-time data from the one or more monitoring devices, via an artificial intelligence engine.

9. The computer program product of claim 8, wherein the computer executable instructions cause the computer processor to in response to identifying occurrence of the potential real-time event, trigger one or more actions.

10. The computer program product of claim 9, wherein the one or more actions comprises at least one of:
automatically controlling one or more entity systems within a facility associated with the entity;
automatically generating and transmitting one or more alerts to the one or more third party entities;
automatically transmitting the real-time data from the one or more monitoring device to the one or more third party entities; and
controlling network associated with network communications of the at least one user device and the one or more entity systems located within the facility.

11. The computer program product of claim 8, wherein identifying the occurrence of the potential real-time event comprises:
identifying one or more patterns that are similar to historical patterns from at least one of (i) data from the at least one user device and (ii) the real-time data from the one or more monitoring devices, wherein the historical patterns are associated with historical events.

12. The computer program product of claim 11, wherein the one or more patterns are associated with type of movements of entity users and the at least one user, type of clothing, type of activity, and type of biosensing signals from the entity users and the at least one user.

13. The computer program product of claim 8, wherein the computer executable instructions cause the computer processor to:
identify that the at least one user device is not within the predetermine distance; and
automatically disconnect the at least one user device from the beacon.

14. The computer program product of claim 8, wherein the one or more monitoring devices comprises at least one of one or more sensors, biosensors, and activity trackers.

15. A computer implemented method monitoring and identifying real-time indicators and patterns to predict occurrence of real-time events, wherein the method comprises:
continuously gathering and monitoring real-time data from one or more monitoring devices associated with an entity;
identifying that at least one user device is within a predetermined distance and automatically transmit a prompt to the at least one user device to connect to a beacon;
identifying that at least one user associated with the at least one user device has connected to the beacon;
continuously monitoring the at least one user device in real-time;
identifying occurrence of a potential real-time event based on continuously monitoring the at least one user device and the real-time data from the one or more monitoring devices, via an artificial intelligence engine.

16. The computer implemented method of claim 15, wherein the method further comprises:
in response to identifying occurrence of the potential event, trigger one or more actions.

17. The computer implemented method of claim 16, wherein the one or more actions comprises at least one of:
automatically controlling one or more entity systems within a facility associated with the entity;
automatically generating and transmitting one or more alerts to the one or more third party entities;
automatically transmitting the real-time data from the one or more monitoring device to the one or more third party entities; and
controlling network associated with network communications of the at least one user device and the one or more entity systems located within the facility.

18. The computer implemented method of claim 15, wherein identifying the occurrence of the potential event comprises:
identifying one or more patterns that are similar to historical patterns from at least one of (i) data from the at least one user device and (ii) the real-time data from the one or more monitoring devices, wherein the historical patterns are associated with historical events.

19. The computer implemented method of claim 18, wherein the one or more patterns are associated with type of movements of entity users and the at least one user, type of clothing, type of activity, and type of biosensing signals from the entity users and the at least one user.

20. The computer implemented method of claim 15, wherein the method further comprises:
identify that the at least one user device is not within the predetermine distance; and
automatically disconnect the at least one user device from the beacon.
